(19) United States
(12) Patent Application Publication
(10) Pub. No.: US 2017/0105852 A1
(43) Pub. Date: Apr. 20, 2017

(54) Connector between a prosthetic socket and a prosthetic limb

(71) Applicant: CHABLOZ COMPOSANTS, Seyssinet-Pariset (FR)

(72) Inventor: Pierre CHABLOZ, Saint Georges de Commiers (FR)

(73) Assignee: CHABLOZ COMPOSANTS, Seyssinet-Pariset (FR)

(21) Appl. No.: 15/187,353

(22) Filed: Jun. 20, 2016

(30) Foreign Application Priority Data
Jun. 18, 2015 (FR) 1555578

(51) Int. Cl. A61F 2/80

(52) U.S. Cl. CPC A61F 2/80; A61F 2002/5083

(57) ABSTRACT

Connector between a prosthetic socket and a prosthetic limb, the prosthetic socket including an open proximal end intended to receive a sleeve, and a closed distal end, the prosthetic limb including a fastener provided with a threading, the connector including: a base; an upper wall extending from the base, and configured to receive the distal end of the prosthetic socket; a tapping formed in the base, and configured to cooperate with the threading of the fastener of the prosthetic limb; wherein the upper wall forms an enclosure configured to envelop the distal end of the prosthetic socket, and wherein the enclosure is off-centered with respect to the base.

### TECHNICAL FIELD

The present invention relates to a connector between a prosthetic socket and a prosthetic limb, to a prosthetic assembly comprising such a connector, as well as to a prosthesis comprising such a prosthetic assembly. Such a connector is particularly suited for a test or temporary socket.

### BACKGROUND ART

A known connector of the state of the art, illustrated in FIG. 1a to 1c, is a connector 1 between a prosthetic socket 2 and a prosthetic limb 3, prosthetic socket 2 comprising an open proximal end intended to receive a sleeve and a closed distal end 20, prosthetic limb 3 comprising a fastener 30 provided with a threading,

the connector comprising:
reception means capable of receiving distal end 20 of prosthetic socket 2, and comprising a base 4;
a tapping (not shown) formed in base 4, and capable of cooperating with the threading of fastener 30 of prosthetic limb 3.

The reception means comprise three lugs 40 made of a metallic material, generally, a stainless steel, extending from base 4. Lugs 40 are distributed around base 4 according to an angle close to 90°. Lugs 40 are manually deformable by means of claws. Lugs 40 are glued to prosthetic socket 2 on a transfer table, and reinforced by means of carbon sheets 5 comprising carbon fibers impregnated with a thermosetting or thermoplastic matrix.

The assembly of lugs 40 to prosthetic socket 2 is long, in the order of one hour, and unsightly due to distal end 20 of socket 2 which remains visible, as well as carbon sheets 5. Further, the connector, made of stainless steel, is relatively heavy.

### SUMMARY OF THE INVENTION

Thus, the present invention aims at overcoming all or part of the previously-mentioned disadvantages, and relates for this purpose to a connector between a prosthetic socket and a prosthetic limb, the prosthetic socket comprising an open proximal end intended to receive a sleeve and a closed distal end, the prosthetic limb comprising a fastener provided with a threading,

the connector comprising:
reception means capable of receiving the distal end of the prosthetic socket, and comprising a base;
a tapping formed in the base, and capable of cooperating with the threading of the fastener of the prosthetic limb;
   the connector being remarkable in that the reception means comprise an upper wall extending from the base, and in that the upper wall forms an enclosure capable of enveloping the distal end of the prosthetic socket.

Thus, a connector according to the invention, due to such an upper wall, enables both to:
conceal the distal end of the prosthetic socket via the enclosure formed by the upper wall, and this for reasons of appearance;
increase the contact surface area of the upper wall which envelopes the distal end of the prosthetic socket, and this to allow a simpler and faster gluing than in the state of the art.

“Proximal” means the end which is located at closest to the residual limb, the residual limb being intended to be enveloped by the sleeve.

“Distal” means the end which is most remote from the residual limb.

“Upper” means that the wall extends above the base in condition of use.

Advantageously, the base comprises a threading extending around the tapping, said threading being capable of cooperating with a locking member, preferably of nut type, capable of locking the rotation of the fastener of the prosthetic limb.

Advantageously, the reception means are made of a plastic material, the plastic material being preferably based on polyamide.

Thus, the selection of a plastic material enables to lighten the connector with respect to the state of the art and to decrease manufacturing costs. The particular selection of polyamide is particularly advantageous since it has a lifetime of at least 15 years in normal conditions of use, and resists a hostile outer environment (projection of water, immersion, corrosion, heat).

Advantageously, the connector is one-piece by molding of the plastic material.

Thus, such a connector is simple to form, requiring no inserts for the threading and the tapping of the base.

Advantageously, the upper wall comprises a layer of a two-component sealant arranged to assemble the upper wall to the distal end of the prosthetic socket.

Thus, such a layer enables to rapidly glue, for in the order of 5 minutes, the connector to the socket, and robustly, due to the significant contact surface area of the upper wall which envelops the distal end of the prosthetic socket.

According to a feature, the upper wall has at least one transverse section forming a closed curve.

According to a feature of the invention, the upper wall is arranged so that the enclosure is off-centered with respect to the base.

In the case of a prosthetic knee, the load line should generally run in front of the articulation center (or of the articulation axis) of the knee to obtain a prosthetic knee locked in extension. As illustrated in FIG. 9, in front cross-section view, load line LC runs through the middle of the hip, the middle of the knee, and the middle of the ankle. The load line is thus intended to run through a central area of the base. Now, as illustrated in FIG. 10, the articulation center of the knee is not aligned with axis A of the stump (also corresponding to the longitudinal axis of femur F). Thus, an advantage of the off-centered arrangement of the enclosure with respect to the base is to ease the assembly of the connector to the prosthetic socket, taking into account the misalignment between the articulation center of the knee and the axis of the stump. The load line may thus easily run in front of the articulation center of the knee.

However, for certain types of prosthetic knees, the load line is close to the articulation axis of the knee. Now, prosthetic sockets are generally aligned with an approximate 5° flexion. As a result, the load line runs behind the stump axis. In this case, an advantage of the off-centered arrangement of the enclosure with respect to the base is to ease the assembly of the connector to the prosthetic socket, taking into account the misalignment between the load line and the stump axis.

Further, such an off-centered arrangement of the enclosure relative to the base is all the more important as the fastener of the prosthetic limb (term “anchor” is generally used) and the connector are not simultaneously mounted on the prosthetic socket. Indeed, the anchor is mounted subsequently on the prosthetic socket.

According to a feature of the invention, the base has a transverse cross-section having a first center of symmetry; the upper wall has a transverse cross-section having a second center of symmetry; and the first and second centers of symmetry are shifted by a distance along a transverse direction. Said distance is adapted to the misalignment between the articulation center of the knee and the axis of the stump or to the possible misalignment between the load line and the stump axis.

“Transverse” means a direction perpendicular to the load line.

The present invention also relates to a prosthetic assembly comprising
a prosthetic socket comprising an open proximal end intended to receive a sleeve and a closed distal end;
a connector according to the invention, the distal end being enveloped with the enclosure formed by the upper wall.

Advantageously, the prosthetic socket is based on modified polyethylene terephthalate glycol (PET-G) or based on a polyepoxide.

The present invention finally relates to a prosthesis comprising:
a prosthetic assembly according to the invention,
a prosthetic limb, preferably a prosthetic knee, comprising a fastener provide with a threading, the fastener being arranged to cooperate with the tapping to fasten the prosthetic limb to the base.

Advantageously, the base of the connector comprises a threading extending around the tapping, and the prosthesis comprises a locking member, preferably of nut type, arranged to cooperate with the threading of the base to lock the rotation of the fastener of the prosthetic limb in the tapping of the base.

### BRIEF DESCRIPTION OF THE DRAWINGS

The foregoing and other features and advantages will be discussed in detail in the following non-limiting description of different embodiments of the invention, in connection with the accompanying drawings, among which:

FIGS. 1a to 1c (already discussed) are simplified perspective views of a connector according to the state of the art,

FIG. 2 is a simplified side view of a connector according to a first embodiment of the invention,

FIG. 3 is a simplified bottom perspective view of a connector according to the first embodiment,

FIG. 4 is a simplified perspective view of a connector according to a second embodiment of the invention,

FIG. 5 is a simplified cross-section view of a connector according to the second embodiment of the invention,

FIG. 6 is a simplified side view of a nut capable of fitting a connector according to the invention,

FIG. 7 is a simplified perspective view of a connector according to a third embodiment,

FIG. 8 is a simplified perspective view of the connector illustrated in FIG. 7,

FIG. 9 is a simplified front view of a prosthetic assembly of the state of the art, illustrating the load line,

FIG. 10 is a simplified sagittal view of a stump illustrating the misalignment between the articulation center of the knee and of the stump axis.

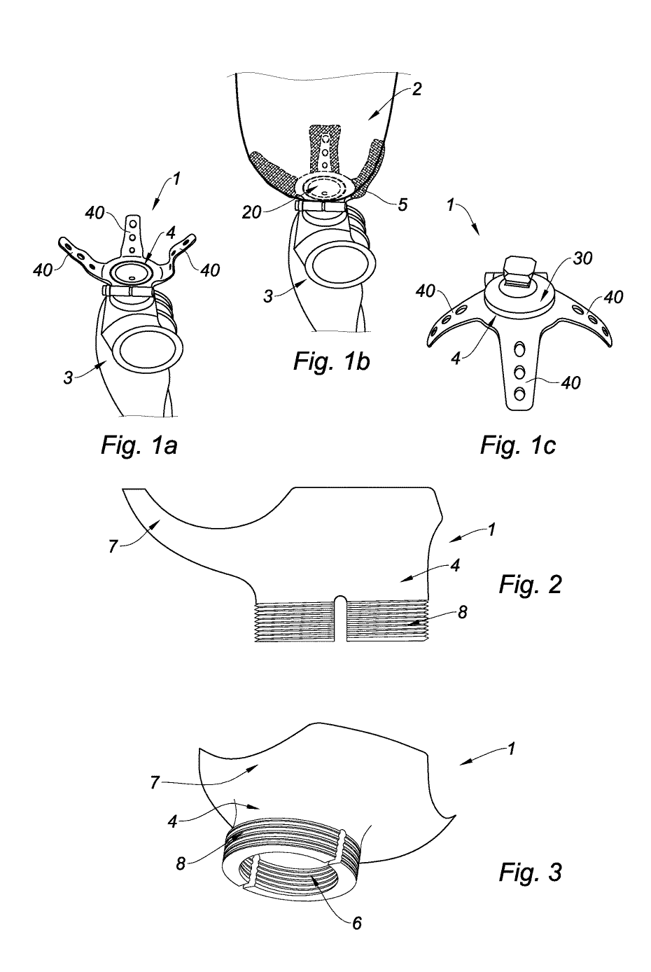

For the different embodiments, the same references will be used for identical elements or elements performing the same function, to simplify the description. The technical characteristics described hereafter for different embodiments are to be considered separately or according to any technically possible combination.

### DETAILED DESCRIPTION OF THE EMBODIMENTS

The connector illustrated in FIGS. 2 to 5 is a connector 1 between a prosthetic socket (not shown) and a prosthetic limb (not shown), the prosthetic socket comprising an open proximal end intended to receive a sleeve and a closed distal end, the prosthetic limb comprising a fastener provided with a threading. The prosthetic socket is advantageously based on modified polyethylene terephthalate glycol (PET-G) or based on a polyepoxide. The prosthetic limb is preferably a prosthetic knee. The sleeve is intended to envelop the residual limb

Connector 1 comprises:
reception means capable of receiving the distal end of the prosthetic socket, and comprising a base 4;
a tapping 6 formed in base 4, and capable of cooperating with the threading of the fastener of the prosthetic limb.

The reception means comprise an upper wall 7 extending from base 4. Upper wall 7 forms an enclosure capable of enveloping the distal end of the prosthetic socket. The enclosure formed by upper wall 7 comprises a concave inner surface 70, preferably closed, and capable of enveloping the distal end of the prosthetic socket. Upper wall 7 advantageously has at least a transverse cross-section, for example, substantially circular, forming a closed curve. Upper wall 7 is advantageously arranged so that the formed enclosure is off-centered with respect to base 4.

Upper wall 7 advantageously comprises a layer of a two-component sealant (not shown) arranged to assemble upper wall 7 to the distal end of the prosthetic socket, the two-component sealant being preferably based on polyurethane.

The reception means are advantageously made of a plastic material, the plastic material being preferably based on polyamide. In other words, base 4 and upper wall 7 are advantageously made of plastic material. Connector 1 is advantageously one-piece by molding of the plastic material, preferably by injection. In other words, base 4. upper wall 7, and tapping 6 are advantageously one-piece by molding of the plastic material.

In an embodiment, base 4 advantageously comprises a threading 8 extending around tapping 6, said threading 8 being capable of cooperating with a locking member 9 (illustrated in FIG. 6), preferably of nut type, capable of locking the rotation of the fastener of the prosthetic limb. Base 4, upper wall 7, tapping 6, and threading 8 are advantageously one-piece by molding of the plastic material.

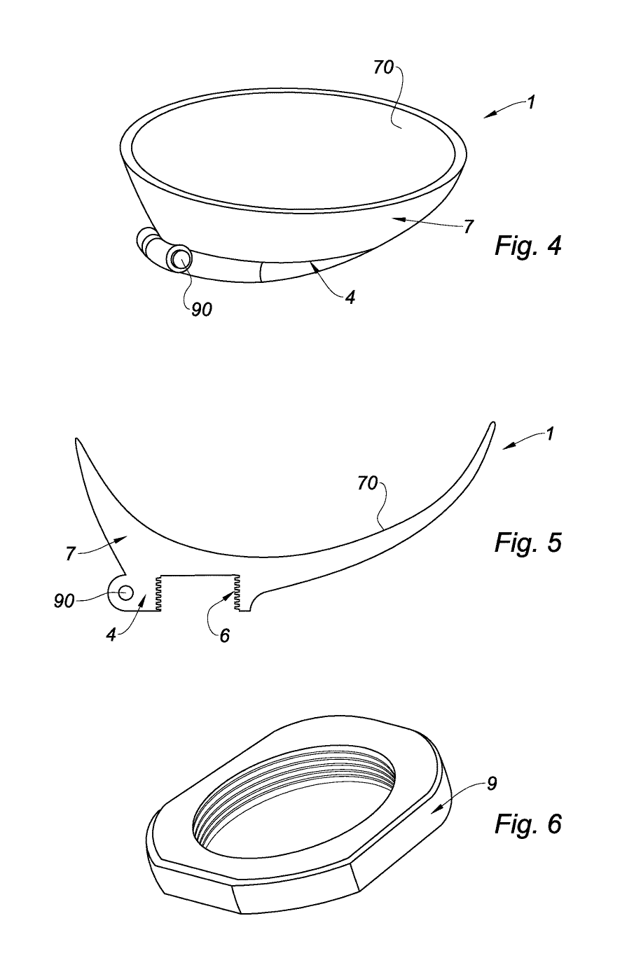

In an alternative embodiment illustrated in FIGS. 4 and 5, the locking of the rotation of the fastener of the prosthetic limb is ensured by a hexagon socket screw 90, preferably BTR.

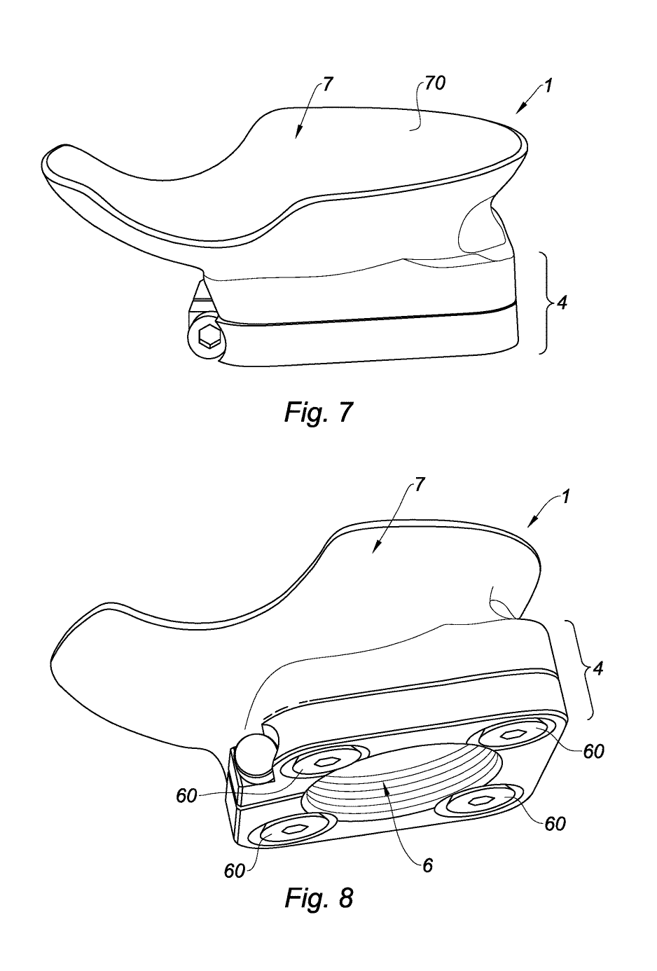

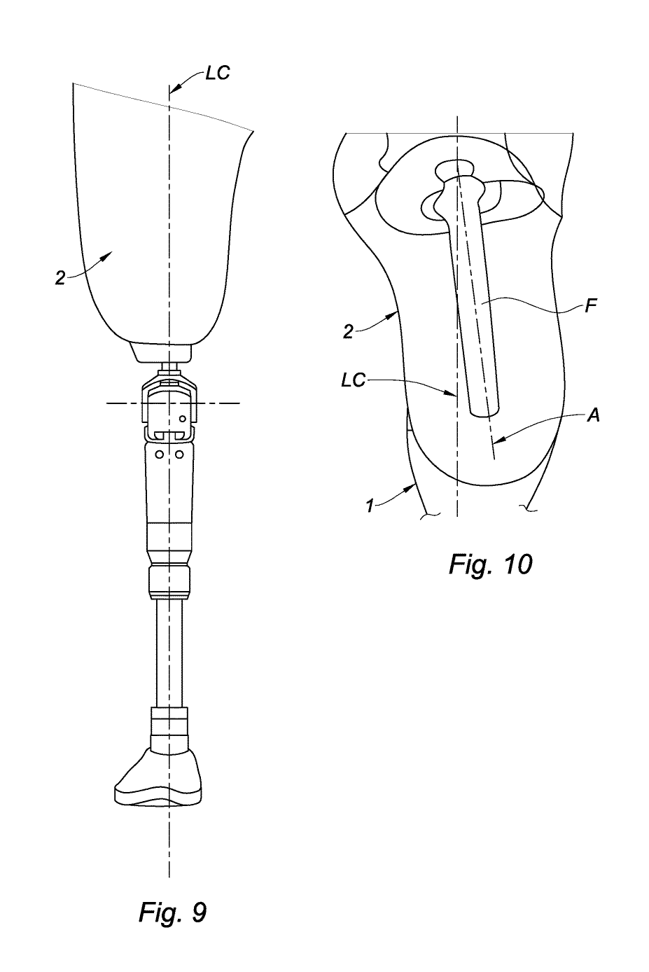

In the embodiment illustrated in FIGS. 7 and 8, base 4 of connector 1 comprises:
a first portion, preferably made of a plastic material;
a second portion, preferably of metal insert type, provided with a central tapping 6. Central tapping 6 is intended to cooperate with fastener 30 to fasten prosthetic limb 3 to the second portion of base 4.

Upper wall 7 extends from the first portion of base 4. The second portion of base 4 preferably comprises four peripheral tappings formed around central tapping 6. The second portion of base 4 may be fastened to the first portion of base 4 by four fastening screws 60 cooperating with the four peripheral tappings. The peripheral tappings preferably have an oblong transverse cross-section, for example, oval-shaped, to allow adjustments in translation of the second portion of base 4.

1. A connector between a prosthetic socket and a prosthetic limb, the prosthetic socket including an open proximal end intended to receive a sleeve, and a closed distal end, the prosthetic limb including a fastener provided with a threading, the connector including:
a base;
an upper wall extending from the base, and configured to receive the distal end of the prosthetic socket;
a tapping formed in the base, and configured to cooperate with the threading of the fastener of the prosthetic limb;
wherein the upper wall forms an enclosure configured to envelop the distal end of the prosthetic socket, and wherein the enclosure is off-centered with respect to the base.

2. The connector according to claim 1, wherein the base includes a threading extending around the tapping, said threading being configured to cooperate with a locking member capable of locking the rotation of the fastener of the prosthetic limb.

3. The connector according to claim 1, wherein the base and the upper wall are made of a plastic material based on polyamide.

4. The connector according to claim 1, wherein it is one-piece by molding of a plastic material.

5. The connector according to claim 1, wherein the upper wall includes a layer of a two-component sealant arranged to assemble the upper wall to the distal end of the prosthetic socket.

6. The connector according to claim 1, wherein the upper wall has at least one transverse cross-section forming a closed curve.

7. A prosthetic assembly including:
a prosthetic socket including an open proximal end intended to receive a sleeve, and a closed distal end;
the connector according to claim 1, the closed distal end being enveloped by the enclosure formed by the upper wall.

8. The prosthetic assembly according to claim 7, wherein the prosthetic socket is based on modified polyethylene terephthalate glycol (PET-G) or based on a polyepoxide.

9. A prosthesis including:
the prosthetic assembly according to claim 7;
a prosthetic limb including a fastener provided with a threading, the fastener being arranged to cooperate with the tapping to fasten the prosthetic limb to the base.

10. The prosthesis according to claim 9, wherein the base of the connector includes a threading extending around the tapping, and wherein the prosthesis includes a locking member arranged to cooperate with the threading of the base to lock the rotation of the fastener of the prosthetic limb in the tapping of the base.
